(19) United States
(12) Patent Application Publication
(10) Pub. No.: US 2010/0034873 A1
(43) Pub. Date: Feb. 11, 2010

(54) TRANSDERMAL RICINOLEIC ACID COMPOSITIONS

(76) Inventor: Keith Delprete, Coopersburg, PA (US)

Correspondence Address: BLANK ROME LLP, WATERGATE, 600 NEW HAMPSHIRE AVENUE, N.W., WASHINGTON, DC, 20037, US

(21) Appl. No.: 12/186,825

(22) Filed: Aug. 6, 2008

(51) Int. Cl. A61K 9/127; A61K 31/20; A61K 33/38; A61K 33/34; A61K 33/32; A61K 33/26; A61K 33/06; A61K 33/04; A61P 25/06; A61P 19/02; A61P 25/16; A61P 31/10; A61P 31/04

(52) U.S. Cl. 424/450; 514/560; 424/619; 424/630; 424/639; 424/641; 424/646; 424/682

(57) ABSTRACT

Transdermal compositions containing castor oil are described. The compositions comprise ricinoleic acid and a transdermal vehicle with optional additional agents. One optional additional agent is an adrenergic drug which may cause the ricinoleic acid to penetrate the skin more deeply and have a longer lasting effect. The compositions can be used for treating a variety of ailments traditionally treated with castor oil or castor oil packs, without the mess and inconvenience of using castor oil packs.

### FIELD OF THE INVENTION

The present invention relates to compositions comprised of ricinoleic acid, the main component of castor oil, one or more transdermal vehicles and optional other agents. The compositions can be used for treatment of ailments traditionally treated by application of castor oil and castor oil packs.

### BACKGROUND OF THE INVENTION

Castor oil, along with ricinoleic acid and its salts have been used as a folk medicine remedy for hundreds of years for a wide variety of ailments. Folk medicine literature describes castor oil taken internally for the treatment of gastrointestinal problems or applied directly to the skin for the treatment of surface infections. Castor oil is also thought to improve lymphatic flow and liver function when applied in heated castor oil packs for extended periods of time.

Castor oil absorbed through the skin is thought to improve a variety of ailments associated with the liver, the lymph system, and the nervous system, including liver cirrhosis, liver congestion, hepatitis, gallbladder diseases, arthritis, fluid retention and swelling, swollen lymph nodes, colon problems such as Crohn's disease or colitis and a broad series of both bacterial and viral infections. Further indications are discussed in Williams, Alternatives, 1995, volume 6, number 1.

To cause castor oil to be readily absorbed through the skin, it is traditionally applied in a castor oil pack. Typically, a cloth is soaked with castor oil and applied to the area to be treated. To prevent the castor oil from leaking and staining other materials such as clothing, the cloth is typically covered with plastic wrap or a garbage bag. A heating pad is then applied over the pack to enhance the penetration of castor oil across the skin. Most castor oil pack protocols require keeping the pack in place and the heating pad applied for at least an hour, at least twice a day to achieve the desired effect.

Although castor oil packs provide a way to obtain the benefits of ricinoleic acid when absorbed through the skin, the packs are messy and require thorough preparation to apply. Further, the subject is forced to remain stationary for considerable amounts of time as the packs must be heated in place for long enough to allow the castor oil to penetrate the skin. As such, there remains a need in the art for compositions containing ricinoleic acid that allow simple and quick application.

### SUMMARY OF THE INVENTION

It is an object of the present invention to provide transdermal compositions for treating an ailment comprising ricinoleic acid and a transdermal vehicle. The transdermal compostions of the present invention can be used for treating ailments typically treated with topically applied castor oil or castor oil packs. The transdermal compositions of the present invention are rapidly and cleanly absorbed into the skin, providing the therapeutic benefits of ricinoleic acid without the mess, time and inconvenience of castor oil packs.

It is a further object of the present invention to provide transdermal compositions for treating an ailment comprising ricinoleic acid, a transdermal vehicle and an adrenergic drug. The presence of the adrenergic drug may cause the ricinoleic acid to penetrate more deeply, giving a longer lasting effect.

It is a yet a further object of the present invention to provide methods for treating ailments traditionally treated with topically applied castor oil or castor oil packs. The methods of the present invention comprise application of a transdermal composition comprising castor oil and a transdermal vehicle to the subject in an area of the subject's body relative to the ailment to be treated.

### DETAILED DESCRIPTION OF THE INVENTION

In one embodiment, the present invention provides compositions with ricinoleic acid and a transdermal vehicle that can be used to quickly and simply administer ricinoleic acid treatments to a subject. The compositions may be administered to the skin in an appropriate area, whereby they be readily absorbed through the skin, without the need for heating. Further, as the compositions are readily absorbed, they leave little or no residue which may stain clothing or cause an uncomfortable feel for the subject.

The ricinoleic acid is the main component of castor oil, making up about 90% of the fatty acid content of the oil. In certain embodiments, the compounds of the present invention are formulated using castor oil to form a compound comprising ricinoleic acid. However, it is also contemplated that the compounds can be formed using other sources of ricinoleic acid and its salts, including sources that have a higher concentration of ricinoleic acid than is typically found in castor oil. In all cases where ricinoleic acid is used in forming the compositions of the invention, it should be apparent that that salts of ricinoleic acid may also be used.

The castor oil used in the compositions of the present invention may be any type of castor oil typically used in castor oil therapies. Although castor oil of a high purity will typically be used, castor oils that are less than 100% pure, including castor oils with amounts of additional oils or other compounds may be used. In certain embodiments of the invention, the castor oil used is cold-pressed castor oil.

A large variety of transdermal vehicles known in the art may be used in the compositons of the present invention. In certain embodiments of the invention, the transdermal vehicle contains lecithin. As example of a transdermal vehicle contemplated for use in the present invention is lecithin organogel as described in U.S. Pat. No. 5,654,337 to Roentsch et al., the disclosure of which is hereby incorporated by reference herein. Lecithin organolgels are transdermal vehicles which are clear, thermostable, pharmaceutically acceptable gels that allow for the prolonged storage of active agents without loss in activity.

It is also contemplated that other transdermal vehicles can be used. One type of transdermal vehicles that may be used are liposomes and vesicles, including phospholipids, transferosomes and solid lipid nanoparticles. The transferosomes used with the present invention may include mixtures of phospholipids, surfactants and solvents as are well known in the art.

Additional transdermal vehicles contemplated include those including fatty acids and their esters, such as oleic acid; terpenes, such as limonene; surfactants such as polysorbate (TWEEN) and sodium dodecyl sulfate; and solvents such as DMSO and ethanol. Chemical penetration enhancers, such as AZONE (lauracapram) and its derivatives, may be used in the transdermal agents. Examples of Azone derivatives are described in Benson et al., Current Drug Delivery, 2005, 2: 23-33. Still other chemical penetration enhancers are contemplated, such as SEPA (2-n-nonyl-1,3-dioxolane) and its derivatives, which are described in U.S. Pat. No. 4,861,764 to Samour et al., the disclsosure of which is hereby incorporated by reference herein.

Examples of transdermal vehicles suitable for use in the present invention include those transdermal systems described by Benson et al., mentioned above; Prausnitz et al., Nature Rev. Drug Discovery, 2004, 3:115-124; Maibach and Smith (eds.) Percutaneous Penetration Enhancers, CRC Press, Boca Raton, Fla., 1995; and Gosh et al. (eds.), Chemical Means of Transdermal Drug Permeation Enhancement in Transdermal and Topical Drug Delivery Systems, Interpharm Press Inc, Buffalo Grove, Ill., the disclosure of which are all hereby incorporated by reference.

The transdermal vehicles of the present invention may contain various amounts of agents which promote penetration of the skin. For example, a transdermal vehicle containing AZONE may not be 100% AZONE, but may include additional solvents or surfactants. The transdermal vehicles of the present invention will typically be formulated as is known in the art.

In other embodiments, the transdermal agents of the present invention may be any lotion that does not leave residues on the skin after application. These lotions include lotions known in the art such as oil in water and water in oil emulsions. Examples of lotions contemplated by the present invention include those described in Remington: The Science and Practice of Pharmacy, 21^{st} Edition, published by Lippincott Williams & Wilkins in 2005, particularly Chapter 39 “Solutions, Emulsions, Suspensions and Extracts.”

In other embodiments, the compositions of the present invention contain additional agents. In certain embodiments, the compositions of the present invention may contain an adrenergic drug. In general, adrenergic drugs are compounds that stimulate the adrenergic nerves directly by mimicking the action of norepinephrine or indirectly by stimulating the release of norepinephrine. Non-limiting examples of adrenergic drugs contemplated by the invention include phenylephrine, epinephrine, norepinephrine, phenylpropanolamine, ephedrine, pseudoephedrine, and oxymetcoline. Without wishing to be bound by theory, the adrenergic drug may increase the penetration of the oil, causing it to have a longer lasting effect than in compositions lacking an adrenergic agent.

In certain other embodiments, the compositions of the present invention may contain guaifenesin. Guaifenesin (CAS 93-14-1) is a commercially available compound with the chemical name 3-(2-methoxyphenoxy)-1,2-propanediol and the chemical formula C₁₀H₁₄O₄. Without wishing to be bound by theory, guaifenesin may act as a Theological agent to thin out the compositions of the present invention, and hence, lower their viscosity.

The optional additional agents may also include therapeutic agents known in the art. These therapeutic agents may be metals or metal salts such as copper, magnesium, manganese, selenium, sodium, potassium, zinc, nickel, cobalt and iron, or the metal salts thereof. The therapeutic agents may be homeopathic remedies, such as those described in A Dictionary of Practical Materia Medica by John Henry Clarke, published by the Homeopathic Publishing Company in 1902. In certain embodiments, the homoepathic agents used may be kali muriaticum (potassium chloride), sulphur, agentum nitricum (silver nitrate), hippozenum, and mixtures thereof. Additionally, the therapeutic agents may also be herbal remedies that are well known in the art. They may also be drug compounds well known in the art.

The compositions of the present invention may include agents for adjusting the pH of the composition, such as citric acid, hydrochloric acid, sodium hydroxide, potassium hydroxide and other acids and bases known in the art. When used, the preferred agent for adjusting pH is citric acid. It is also contemplated that the compositions of the present invention may contain other additives, such as fragrances, sunscreens and skin softeners such as aloe.

The compositions of the present invention may be formulated in a variety of concentrations. The compositions contain an amount of castor oil effective to relieve the specific symptom being treated. Preferably, castor oil is present at a concentration of about 30% to about 75% of the weight of the final composition. If no other agents are present, the transdermal vehicle will make up the remaining weight of the final composition.

When the transdermal vehicle is a lecithin organogel, it may include the following ingredients in preferable concentrations of the weight of the final composition:

lecithin—about 5% to about 30%;

surfactant—about 5% to about 30%;

urea—about 5 % to about 20%;

water—the balance of the composition.

Preferably, the surfactant of the transdermal vehicle is isopropyl myristate or isopropyl palmitate, which may both be present in the composition. However, other surfactants are contemplated by the invention, such as docusate sodium, Polysorbate 80, glycerin, polyethylene glycol, steatric acid, cetyl alcohol, stearyl alcohol and the like. These other surfactants may be used in combination or alone so that the final concentration of surfactant in the composition is about 5% to about 30% of the weight of the final composition.

If a chemical penetration enhancer such as Azone or Sepa is used, they will typically be present at concentration of about 1% to about 25% of the weight of the final composition. If surfactants or solvents are used, they may be present up to the balance of the weight of the final composition including the castor oil and any other agents. It is contemplated that chemical penetration enhancers can be added to compositions along with solvents and surfactants. It is also contemplated that the concentration of chemical penetration enhancers, solvents and surfactants may not complete the balance of the final composition. In such cases where the balance of the final composition is not completed by the transdermal vehicle, it may be completed by addition of an appropriate amount of water or other liquid so that the final concentrations of the components of the composition are correct.

When present, the preferable concentrations of adrenergic drug in the compositions of the present invention are about 0.001% to about 0.1% of the weight of the final composition. When phenylephrine or epinephrine are present, it is most preferable that they are present at a concentration of about 0.005% to about 0.05% of the weight of the final composition. When present, the preferable concentration of guaifenesin in the compositions of the present invention is about 0.01% to about 30% of the weight of the final composition. More preferably, guaifenesin is present at about 8% to about 12% of the weight of the final composition. When present, the preferable concentration of therapeutic agents in the compounds of the present invention is about 10⁻¹⁰% to about 30% of the weight of the final composition. The preferable concentration for a specific therapeutic agent will vary based on the effectiveness and toxicity of the agent.

If desired, an adrenergic drug is then dissolved, along with any necessary salts or pH adjusting agents, in hot purified water (between 70 and 75° C.). While the adrenergic drug solution is still hot, it is added to the surfactant/solvent/emulsifier mixture and more surfactant is added if necessary.

The compositions of the present invention may be used to treat any ailment commonly treated through the topical application of castor oil and/or castor oil packs. Such ailments include, but are not limited to: lymphatic congestion; swollen lymph nodes; headache; migraine headache; sore throat; chronic fluid retention with swollen joints and pain; arthritis; upper respiratory infections involving the sinuses, tonsils and inner ear; colon problems like Crohn's disease or colitis; gallbladder disease; boils; liver cirrhosis, hepatitis, enlargement or congestion; menstrual-related congestion; appendicitis; hyperactivity; constipation, bowel impaction or adhesions; bladder and vaginal infections; muscle strains; ligament sprains; immune system depression related to HIV/AIDS; nerve inflammations such as sciatica and shingles; Parkinson's disease; multiple sclerosis and cerebral palsy. The compositions of the present invention may also be used to treat ailments on the surface of the skin, including, but not limited to: skin keratosis; ringworm; fungal and bacterial infections; cuts and wounds; abdominal stretch marks (prevention); bursitis; sebaceous cysts; warts; senile lentigo (“liver” or “aging” spots); and itching.

The effective amount of castor oil necessary may vary depending on the ailment to be treated. The compositions of the present invention may be applied once, twice or several times a day to achieve the desired effect. As castor oil is not known to be harmful when applied externally, the compositions can be used frequently for a period of days, weeks, months or even longer without great risk of side effects. When present, the adrenergic drug may cause the castor oil to penetrate deeper into the body, possibly providing a longer lasting effect and requiring less frequent dosage.

The final composition may then be applied as necessary to the part of the body to be treated. Application typically involves use of a roll-on applicator, a spray bottle, a pump bottle, a brush, a gauze, a tissue or the fingers. The compositions may be applied in a thin layer on the skin covering the area to be treated. The area may then be covered with a bandage or gauze, or may be left uncovered. However, because the compositions are formulated to be quickly absorbed and leave no residue on the skin, it is not necessary that they be covered. It is further contemplated that the compositions of the present invention may be integrated directly into packaged bandages for convenient application. The bandages of the invention come with the compositions of the invention pre-applied to allow for easy administration to a subject.

The compositions of the present invention may be stored in a glass jar with a roll-on applicator. It is also contemplated that the compositions may be stored in other containers, including various types of jars and bottles made of glass, Plexiglas, plastic or other polymeric substance. In an alternative embodiment of the invention, the composition is stored in a spray bottle for application by spraying onto the skin.

The embodiments and examples described herein are meant to give non-limiting examples of the compositions and methods of the present invention. It should be understood that there are other embodiments not specifically set forth above that fall within the spirit and scope of the invention as claimed.

### EXAMPLES

### Example 1

### Formation of a Transdermal Ricinoleic Acid Composition

A lecithin/phenylephrine transdermal vehicle was prepared in two steps.

### Step 1: Lecithin/Isopropyl Palmitate Solution

| | 220 mL | |
|---|---|---|
| Lecithin Soya Granular | 100 | g |
| Isopropyl Palmitate, NF, Cosmetic Grade | 117 | mL |
| Sorbic Acid, NF, Cosmetic Grade | 0.66 | g |

The lecithin soya granular and sorbic acid were dispersed in isopropyl palmitate and allowed to stand at room temperature until all particles were dissolved and a clear product was formed.

### Step 2: Gel Base

| | 100 g | |
|---|---|---|
| Polysorbate 80 NF | 10 | g |
| Lecithin/Isopropyl Palmitate Solution (from Step 1) | 22 | mL |
| Docusate Sodium, USP 85% (15% Sodium Benzoate) | 10 | g |
| Urea, USP | 10 | g |
| Phenylephrine Hydrochloride, USP | 10 | mg |
| Purified Water, USP | 45 | mL |
| Citric Acid, USP Hydrous Powder | 2.5 | g |
| Isopropyl Myristate NF or Isopropyl Palmitate NF | 2.8 | mL |

The Polysorbate 80, docusate sodium and lecithin/isopropyl palmitate solution were mixed until a creamy and uniform texture was achieved.

B. The urea, citric acid and phenylephrine hydrochloride were dissolved in 45 mL of hot (70 to 75 degree C.) purified water.

C. While the solution from step B is still hot, it was added to the mixture from step A, followed by the addition of 2.8 mL of isopropyl palmitate. The entire mixture was mixed until a clear amber solution formed.

The transdermal vehicle was mixed with castor oil in a 50:50 ratio to form a transdermal ricinoleic acid composition.

### Example 2

A 47 year old woman had symptoms of swollen neck glands and a sore throat. The 50% castor oil mixture described in Example 1 was applied to neck region from just below the ears down into each side of the throat and also to the back of the neck at bedtime. It was observed the next morning that the sore throat and swollen glands had completely disappeared. No side effects were observed and no residue was noticed at the application site. The same results were noted with this person when applied on other occasions.

### Example 3

A 33 year old women had been complaining of suffering with a sore throat for 3 days without resolution after trying various OTC medications. The 50% castor oil mixture described in Example 1 was applied to the neck area at bedtime. The next morning the sore throat was completely gone. There were no side effects noted or residue.

### Example 4

A 48 year old male was experiencing a sore throat and applied the 50% castor oil mixture described in Example 1 to the throat area at bedtime. The next morning the sore throat was complete gone. There were no side effects noted or residue.

### Example 5

A 47 year old woman complained of pain in the gall bladder region. The 50% castor oil mixture described in Example 1 was applied over the area and it was noted that after 2 hours the discomfort had stopped. There was no side effects or residue noted.

### Example 6

A 47 year old woman complained of sinus a headache. The 50% castor oil mixture described in Example 1 was applied to the side of the neck and above the sinuses on the face. It was observed that the headache disappeared after 1 to 2 hours. There was no side effects or residue noted.

### Example 7

A 22 year old male had complained of an extreme sore throat, swollen neck glands with difficulty in talking for 3 days. He was seen by a physician and checked for Strep throat and was told that it was not present. He applied the 50% castor oil mixture described in Example 1 to the neck and later that day he observed that the pain and swelling had disappeared. There were no side effects or residue noted.

What is claimed is:

1. A transdermal composition for treating an ailment comprising:
a transdermal vehicle; and
ricinoleic acid or a salt thereof in an amount effective to treat the ailment.

2. The transdermal composition of claim 1, wherein the transdermal vehicle is a lecithin organogel.

3. The transdermal composition of claim 1, wherein the transdermal vehicle is selected from the group consisting of: lotions; liposomes; vesicles; phospholipids; transferosomes; solid lipid nanoparticles; fatty acids; fatty acid esters; oleic acid; terpenes; limonene; surfactants; polysorbate; sodium dodecyl sulfate; DMSO; ethanol; lauracapram and its derivatives; and 2-n-nonyl-1,3-dioxolane and its derivatives; and mixtures thereof.

4. The transdermal composition of claim 1, further comprising an adrenergic drug.

5. The transdermal composition of claim 5, wherein the adrenergic drug is selected from the group consisting of: phenylephrine, epinephrine, norepinephrine, phenylpropanolamine, ephedrine, pseudoephedrine, and oxymetcoline.

6. The transdermal composition of claim 1, further comprising guaifenesin.

7. The transdermal composition of claim 1, further comprising a therapeutic agent.

8. The transdermal composition of claim 7, wherein the therapeutic agent is selected from the group consisting of: copper, magnesium, manganese, selenium, sodium, potassium, zinc, nickel, cobalt and iron, and pharmaceutically acceptable salts thereof.

9. The transdermal composition of claim 7, wherein the therapeutic agent is a homeopathic agent.

10. The transdermal composition of claim 9, wherein the homeopathic agent is selected from the group consisting of kali muriaticum, sulphur, agentum nitricum, hippozenum, and mixtures thereof.

11. The transdermal compostion of claim 1, further comprising an additional oil.

12. A transdermal composition for treating an ailment consisting essentially of:
a transdermal vehicle; and
ricinoleic acid in an amount effective to treat the ailment.

13. A transdermal composition for treating an ailment comprising:
a transdermal vehicle in an amount of about 10% to about 70% of the weight of the final composition; and
castor oil in an amount of about 30% to about 75% of the weight of the final composition.

14. The transdermal composition of claim 13, wherein the transdermal vehicle is a lecithin organogel.

15. The transdermal composition of claim 13, wherein the transdermal vehicle is selected from the group consisting of: lotions; liposomes; vesicles; phospholipids; transferosomes; solid lipid nanoparticles; fatty acids; fatty acid esters; oleic acid; terpenes; limonene; surfactants; polysorbate; Sodium Dodecyl Sulfate; DMSO; ethanol; lauracapram and its derivatives; and 2-n-nonyl-1,3-dioxolane and its derivatives; and mixtures thereof.

16. The transdermal compostion of claim 13, further comprising an adrenergic drug in an amount of about 0.001% to about 0.1% of the weight of the final composition.

17. The transdermal composition of claim 16, wherein the adrenergic drug is selected from the group consisting of: phenylephrine, epinephrine, norepinephrine, phenylpropanolamine, ephedrine, pseudoephedrine, and oxymetcoline.

18. The transdermal composition of claim 13, further comprising guaifenesin in an amount of about 0.01% to about 30% of the weight of the final composition.

19. The transdermal composition of claim 13, further comprising a therapeutic agent in an amount of about 10⁻¹⁰% to about 30% of the weight of the final composition.

20. The transdermal composition of claim 19, wherein the therapeutic agent is selected from the group consisting of: copper, magnesium, manganese, selenium, sodium, potassium, zinc, nickel, cobalt and iron, and the pharmaceutically acceptable salts thereof.

21. The transdermal composition of claim 19, wherein the therapeutic agent is a homeopathic agent.

22. The transdermal composition of claim 21, wherein the homeopathic agent is selected from the group consisting of kali muriaticum, sulphur, agentum nitricum, hippozenum, and mixtures thereof.

23. A method for treating an ailment comprising administering to a subject having the ailment a transdermal composition comprising:
a transdermal vehicle; and
ricinoleic acid in an amount effective to treat the ailment.

24. The method of treating an ailment of claim 23, wherein the transdermal vehicle is a lecithin organogel.

25. The method of treating an ailment of claim 23, wherein the transdermal vehicle is selected from the group consisting of: lotions; liposomes; vesicles; phospholipids; transferosomes; solid lipid nanoparticles; fatty acids; fatty acid esters; oleic acid; terpenes; limonene; surfactants; polysorbate; Sodium Dodecyl Sulfate; DMSO; ethanol; lauracapram and its derivatives; and 2-n-nonyl-1,3-dioxolane and its derivatives; and mixtures thereof.

26. The method of treating an ailment of claim 23, wherein the ailment is selected from the group consisting of: lymphatic congestion; swollen lymph nodes; headache; migraine headache; sore throat; chronic fluid retention; arthritis; upper respiratory infections; Crohn's disease; colitis; gallbladder disease; boils; liver cirrhosis, liver hepatitis; liver enlargement; liver congestion; menstrual-related congestion; appendicitis; hyperactivity; constipation, bowel impaction; bowel adhesions; bladder infections; vaginal infections; muscle strains; ligament sprains; immune system depression; nerve inflammation; sciatica; shingles; Parkinson's disease; multiple sclerosis; cerebral palsy; skin keratosis; ringworm; fungal and bacterial infections; cuts; wounds; abdominal stretch marks; bursitis; sebaceous cysts; warts; senile lentigo; and itching.
